(19) United States
(12) Patent Application Publication
(10) Pub. No.: US 2006/0265034 A1
(43) Pub. Date: Nov. 23, 2006

(54) Microwave devices for treating biological samples and tissue and methods for using same

(75) Inventors: Gerard Aknine, Coppet (CH); Jean Daniel Richerd, Nyon (CH)

Correspondence Address: WILEY, REIN & FIELDING, LLP;ATTN: PATENT ADMINISTRATION, 1776 K. STREET N.W., WASHINGTON, DC, 20006, US

(73) Assignee: AMS Medical SA, Nyon (CH)

(21) Appl. No.: 11/396,599

(22) Filed: Apr. 4, 2006

Related U.S. Application Data
(60) Provisional application No. 60/668,073, filed on Apr. 5, 2005.
(60) Provisional application No. 60/668,059, filed on Apr. 5, 2005.
(60) Provisional application No. 60/676,298, filed on May 2, 2005.

(51) Int. Cl. A61F 2/00; A61N 1/00

(52) U.S. Cl. 607/101; 607/156

(57) ABSTRACT

The invention provides Rf and more particularly microwave treatment devices for biological samples or tissue. In a preferred embodiment, the antenna are capable of directing energy to a particularly focussed area under the surface of the skin of a human. In other embodiments, the flexible antenna provides efficient delivery of energy to the sample or tissue regardless of the conformation of the antenna.

### RELATED APLLICATION INFORMATION

This application claims priority benefit of U.S. provisional application 60/668,073 filed on Apr. 5, 2005, 60/668,059, filed Apr. 5, 2005, and 60/676,298 filed May 2, 2005, the entire contents of which are incorporated herein by reference.

### FIELD OF INVENTION

The invention relates to energy emitting devices that can be used to subject samples or living tissue to energy, particularly focused energy capable of heating fluid samples or tissue. In preferred embodiments, the device comprises a patch, annular or concentric antenna device capable of being placed in electrical contact with the sample or tissue. In other preferred embodiments, flexible material is used to sufficiently contact tissue or surface contours without deteriorating the energy delivery or efficiency of the antenna used. In other preferred embodiments, a water or solution-filled bolus chamber encased in a solid material is used. The methods of the invention include: a method to treat skin tissue to reduce or eliminate fine lines, age lines, or wrinkles; a method for treating muscle pain or muscle fatigue for example, after exercise; a method of treating blood samples to reduce or eliminate microorganisms and viruses; and a method for treating, purifying, or decontaminating water or fluid, such as water or fluid used in or circulating through a building, such as in a heating or cooling system. Compared to other probes, electrodes, and antenna used previously, the devices and methods of the invention provide added control and/or focusing potential in emitting energy to advantageously treat or heat specific points under the surface of skin or tissue without adversely effecting nearby tissue. Furthermore, the efficiency of the antenna design allows, in one aspect, the antenna to be shaped to fit over a particular body part or region, thus changing the shape of the antenna, without adversely effecting the direction of the energy emitted or the strength of the field emitted. Accordingly, the invention includes a flexible antenna that efficiently operates when its contour is manipulated during use.

### BACKGROUND OF INVENTION

Microwave applicators take a number of different forms and are used in a number of different treatments. One treatment discussed is heat treatment for superficial disease, which generally requires an applicator capable of treating irregularly shaped tissue or extending into the skin surface to a maximum depth of about 1 cm or more. Previously used microwave applicators operating at either 915 or 433 MHz have demonstrated appropriate penetration of microwaves at a Specific Absorption Rate (SAR) in skin for generating an affect on cell viability. However, these applicators suffer from a number of deficiencies, including inaccurate and inconsistent treatments and results, burning of the skin or tissue, and complex training to avoid these problems in their use. Furthermore, the microwave and radiofrequency approaches that involve surgical or microsurgical placement of antenna or catether-type devices inside the body have obvious disadvantages to those capable of being used in non-invasive methods. Thus, the art desires improved and more effective devices and methods for treating biological samples, skin and tissue, as well as treatments and devices that can be used non-invasively.

### SUMMARY OF THE INVENTION

In one aspect, the present invention provides a generic apparatus or device for treating a fluid, biological sample, living tissue or organs with Rf or microwave energy (the preferred frequency or range of frequencies is between about 200 MHz and about 2.5 GHz, or from about 300 MHz to about 1.2 GHz). In general, the devices can be used to focus microwave energy, or the biological effects of the energy, in defined and/or small areas and heat and/or effect the cells or fluid within the areas to impart some beneficial result or characteristic. In a first specific embodiment of the devices of the invention, and the methods for using the devices, the invention provides a device and method for treating human or animal skin in order to correct disease, improve appearance, reduce or eliminate the signs of aged skin, or reduce or eliminate fine lines or wrinkles in the skin. In this embodiment, the components and elements are selected so that the microwaves can be focused on, for example, the epidermal layer of the skin and the fibers within the epidermal layer that reflect on the level of tightness of the skin or degree of wrinkles present. Thus, fibers in the epidermal layer can be can be severed or denatured as a result of the microwave treatment, whether by thermal effects or other effects. Alternatively, any skin tissue, in particular the underlying collagen fiber layer, such as those of the dermis or superficial dermis, respond to the treatment with a slight and focused damage to the fibrous material and causing it to heal or regenerate. The healing or regenerating tissue will impart an improved, tightened appearance to the surface of the skin, removing wrinkles and fine lines. Thus, desired areas or portions of the skin's surface can be tightened to improve its appearance.

In a second specific embodiment, the invention provides a device and method for treating muscle in a human or animal. The components or elements are selected so that the microwaves can be directed to muscle tissue and optionally conformed against the skin proximate to the muscle tissue. Various holding or sleeve-type apparatus can be used to house a flexible antenna so that, in one example, an optional inflatable region can press the surface of the antenna designed to emit energy against the skin or area proximate to the muscle. These devices and methods can be used for various muscle pains and conditions, and can be used to prepare for or recover from muscle exertion. The flexible substrates used can be selected from many polymer or copolymer compounds with an appropriate dielectric constant. Preferred flexible compounds are silicone polymers, and those having a dielectric constant of about 2.5 to 3.5. In another embodiment, the design, construction, and/or use of a flexible antenna provided by the invention allows an optimization of the efficiency of the antenna. Thus, for example, reflected power is reduced and the emitted or irradiated power to the sample or tissue is maximized. In preferred embodiments, the optimization is present regardless of the conformation of the flexible antenna. Similarly, a system incorporating the invention can detect the reflected power while in use so that a detector or display of the reflected power is presented to the user in order to re-position or re-configure the antenna, or adjust the frequency or power or pulses from the generator, to control and/or maximize the energy delivered to the tissue.

In another specific embodiment, a microwave emitting device and method of using it can be used to treat samples of blood or plasma in order to remove or reduce the level of biological, microbiological, or viral contamination. In another specific embodiment, the invention provides a device and method for treating water or fluid in a building, such as circulating heating/cooling water or other circulating fluid. In this embodiment, the microwave emissions can be used to remove unwanted bacterial and/or viral contamination as the water or fluid passes over or in proximity to the microwave emitting device. Many other embodiments are possible and can be devised or constructed from the basic concepts and specific description presented here.

In another general aspect, the invention comprises a method for using a microwave emitting device, where the device comprises a microwave antenna formed within a substrate and connected to a power amplifier. The antenna design takes into consideration the intended use, the permittivity of the components used, and the material being treated, as wells as the power, wavelength and energy desired for that use. In various examples, the antenna can be referred to or be a concentric array radial line slot antenna, a slot aperture antenna, an annular slot antenna, a patch antenna, a multi-layered concentric aperture antenna, a dual concentric conductor antenna, a coplanar patch antenna, and an array antennae. Examples of these antennae are known to one of skill in the art. Several embodiments are shown in the Drawings, and one embodiment encompasses an antennae consisting of an approximately 8-10 um, or 8-10 mm, in diameter in a concentric array. In the general case, the antenna comprises some sort of substrate within which, or on which, or formed on which is a metallic antenna. The antenna will have a primary microwave emitting surface designed to be placed against skin or tissue, or fabric or clothing covering skin or tissue, to adequately or sufficiently emit energy into the tissue to produce a biological or treatment effect. The antenna can be designed to be used without water, or without aqueous solution or gel between the emitting surface of the antenna, or with water or aqueous solution or gel.

Connected to the substrate on the surface designed to emit microwaves, in one optional embodiment, is an aqueous or solution-filled bolus chamber having a surface for contacting a sample or tissue. In general, the bolus chamber is composed of a solid, semi-rigid, or rigid polymer or copolymer, such as polyoxymethylene, designed with a particular dielectric coefficient in mind for the use desired. The substrate in other embodiments can be a silicone polymer, such one formed into a sheet of about 1.5 mm in thickness and of various hardness and dielectric properties.

The methods of the invention can further comprise, in one aspect, positioning the microwave emitting device proximate to or in contact with a sample or tissue under conditions in which the microwaves can penetrate through the antenna substrate surface or bolus and penetrate the sample or tissue. Typically, a water, solution, gel or moisture layer is applied to the surface of the bolus contacting the sample or tissue, or the sample or tissue or treated with water, a solution, or moisture. The layer of material between the bolus surface where microwaves are emitted and the sample or tissue can affect the ability or efficiency of the microwaves to penetrate the sample or tissue, as known in the art. After the surface of the bolus is properly positioned, which may include treating one or more surfaces as just noted or similarly treating those one or more surfaces, one or more microwave pulses are generated.

In any of the muscle treatment, skin treatment, and/or wrinkle reducing methods, the microwave pulse can be about 1 msec in length and of a desired frequency. A microwave pulse can also be about 1 sec in length or other duration, for example 20 ns to 2 secs, or 20 ns to 30 ms, or 30 ms to 500 ms, or 500 ms to 1 sec. In other embodiments, the pulses can be longer. The microwave pulse can be of a frequency and energy that penetrates through the skin and is capable of penetrating to a desired depth of the skin. For example, the frequency can be 200 MHz to 2.5 GHz, 300 MHz to 1.2 GHz, or 300 MHz to 1 GHz, or 400 MHz to 950 MHz, or 400 MHz to 500 MHz, or 850 MHz to 950 MHz. As discussed, the antenna and generator can be calibrated, tuned, and/or matched at a frequency X but operated at a frequency different from X, for example half X or twice X, or within a range from about half X to about twice X. Typically, the frequency selected for the generator and waveguide for a λ/2 dipole type antenna is twice the frequency desired to be penetrating through the sample or tissue. Thus, in one embodiment, the frequency selected is about 866 MHz for a dual concentric conductor antenna, however one of skill in the art is capable of selecting, testing, and using many other possible frequencies to affect the temperature of tissue, such as one of more of the frequencies and pulse durations listed above (see, for example, Mizushina et al., “Effects of water-filled bolus on the precision of microwave radiometric measurements of temperature in biological structures,” Microwave Symposium Digest, 1900, IEEE MTT International).

In certain embodiments, the antenna is an annular slot antenna of a size capable of using a power transmitter to emit approximately twice the frequency desired for use in the sample or tissue, and wherein the dielectric constant of the substrate is approximately 3.5. The selection of the antenna, frequency, substrate, and the temperature of the solution in the bolus are such that the microwave emitting device is capable of heating the sample or tissue proximate to the surface for contacting the sample or tissue. Furthermore, the elements of the device can be selected to resonate at the desired operating frequency.

In certain embodiments, the bolus is filled with water, deionized water, distilled water, saline solution, or a solution of silicon in water. Similarly, the substrate for certain or any embodiment is a polymer or copolymer, preferably a POM (polyoxymethylene) polymer having a dielectric coefficient of approximately 3.5. In an embodiment where the solution is deionized or distilled water, the temperature of the water in the bolus can be selected from between about 17° C. and about 28° C. The temperature of the solution or water in the bolus can be regulated by circulating the water or solution into or with a temperature controlled bath external to the bolus and/or by using commercially available temperature controlling devices.

In a preferred embodiment, the method encompasses treating the derma layers or epidermal layers of skin and the antenna is selected to focus the emitted microwaves at a point or area approximately 1.5 mm to 2 mm below the external surface of the epidermis or tissue, or approximately 2 mm to 3 mm, or approximately 3 mm to 4 mm, or approximately 4 mm to 5 mm, or a depth of 0.5 mm to 16 mm from the surface of the skin or tissue. The method encompasses a treatment wherein the tissue is skin and the emitted microwaves are directed to an area of the face where aged or wrinkled skin is present, such as around the eyes, around the lips, on the chin, on the neck, and on the forehead. Other areas, such as arms and chest areas, can also be treated.

In other general embodiments of the invention, an Rf or microwave energy emitting device is used, wherein the device is similar to that described above. Preferred energy wavelengths are above about 300 MHz, and 915 MHz can be preferred for embodiments complying with FCC guidelines for medical devices. The energy emitted can be focused to a desired point under the surface of skin or tissue by selecting the frequency transmitted from the generator, the power selected from the generator, the dielectric of the substrate used in the antenna, the impedance of the transmission lines, the configuration of the antenna and the material used in its component parts, as well as the size and presence of a solution-filled bolus and the solution used and the temperature of the solution used. In addition, the bioelectrical impedance or differing dielectric of certain tissues and skin samples, for example, should be considered in the design to optimize energy treatment to a particular tissue of sample of skin.

In other preferred embodiments, a microwave or Rf emitting device and method of using comprise a radiometric, ultrasound, or other monitoring device to sample the conditions and/or temperature and/or temperature change in the tissue or sample, particularly at a desired depth in the sample or tissue. Thus, the methods and devices comprise, in addition to a microwave or Rf emitting device and methods, a non-invasive radiometer, thermometer and/or imaging device as known in the art or available to one of skill in the art. In certain embodiments, a dual mode antenna, or a cylindrical antenna, can be designed for combined use and/or can perform the combined microwave emitting and radiometric receiving functions (see, for example. Jacobsen et al., IEEE Transactions Biomed. Eng. 47: 1500-09 (2000)). In other embodiments, the device can be coupled with or used in conjunction with a sonography device, such as with a 20-MHz B-mode ultrasound scanner (DUB 20S, taberna pro medicum, Lüneburg, Germany).

In another, general embodiment, the invention encompasses a microwave emitting device for treating skin comprising a microwave antenna capable of emitting a directional, focused beam of radiation, wherein the antenna is connected to, formed on, or embedded in a substrate, wherein the substrate has a back surface and a front surface and the direction of the emitted radiation emanates from the front surface. The device further comprises a solution-filled bolus attached to the substrate at the front surface of the substrate, wherein the solution is encased in a solid plastic having a desired dielectric constant and wherein the bolus is about 5 mm in thickness. The antenna is connected to a power source or supply and is capable of sending energy to the antenna resulting in pulses of microwave emissions. The device can further comprise a temperature-controlling device capable of maintaining the bolus at a predetermined temperature. The temperature-controlling device can be operationally coupled to a computer-controlled system for regulating the microwaves emitted in response to the temperature detected in the sample or tissue. In this way, the microwave emitting device can be fine-tuned to emit specific radiation levels to specific depths of a tissue or sample. In general, the frequency and energy of the microwaves capable of being emitted from the antenna are selected based upon the permittivity of the selected substrate, the solution-filled bolus, the solution in the bolus, and the temperature of the bolus, such that the emitted microwaves penetrate through the bolus and enter skin in contact with the bolus to a desired depth to affect the appearance of the skin, or to penetrate a sample, such as plasma, and heat the sample to eradicate or reduce the level of microorganisms present.

In these and various other uses and embodiments of the invention, the methods of the invention can include the step of using the applicator to generate a focused, converging, and/or quasi-transverse electromagnetic surface wave within the tissue or skin by utilizing the differing dielectric and conductivity characteristics of the skin layers and/or muscle layers and/or fat layers. The amplitude and phase for the individual antenna and/or microwave emitting elements on the device are selected to adjust the focal point or area within a target region below the surface of the skin. Simultaneously with the emitting or microwave radiation, the bolus can be used to cool the skin surface to prevent or reduce skin burns and acute pain. Cooling formulations, analgesics or anesthetics, and sprays can also be used, as known in the art.

Other embodiments and advantages of the invention are set forth in part in the description that follows, and in part, will be obvious from this description, or may be learned from the practice of the invention.

### BRIEF DESCRIPTION OF THE DRAWINGS

For a more complete understanding of the invention and some advantages thereof, reference is now made to the following descriptions taken in connection with the accompanying drawings in which:

FIG. 1 shows a cross-section view of an exemplary embodiment of the aspect of a device of the invention that can be placed in contact with skin, tissue, or a sample. Antenna element (1) is formed in a circular aperture or concentric form around the edge. The substrate (3) surrounds and maintains the form of the antenna and is typically prepared of a solid material having a desired dielectric constant. Surface (2) is the back side or side opposite to the direction of the emitted microwaves into skin or tissue or sample, and surface (2) can be coated in metal or other material to prevent energy from being emitted. Bolus (5) is formed on the opposite side with respect to surface (2) and is intended to contact the skin, tissue, or sample involved. Bolus chamber (4) is filled with a solution, preferably deionized water, and the temperature of the solution can be controlled. In some embodiments, changing the temperature of the water or solution within the bolus acts as a focusing effect on the energy emitted into the skin or tissue, such that select temperatures can direct energy to a certain depth and/or area under the surface, and other temperatures can be selected for different depth and/or areas. In a preferred embodiment, the thickness of bolus chamber (4) is about 5 mm when contact with skin is desired.

FIG. 2 shows an exemplary flexible antenna embodiment, where elements shown in FIG. 1 are supported on a flexible surface (11). Supporting members or braces (7) and (10) hold the bolus chamber to a desired depth or thickness (8) and (9) throughout the device. Temperature controlled water or other solution flows through input tube (14) into the bolus, and out through output tube (12). Cable (13) connects energy transmitter to antenna leads. Surface (6) can be coated or treated to reduce emissions through it. As in other embodiments, the antenna design can be selected from many available or known in the art.

FIG. 3 shows an exemplary array antenna embodiment with a bolus of 5 mm in thickness. The elements as noted in the text are shown, including a circulating bolus inlet and outlet to control the temperature of the bolus, a connection to a power source or supply, in the form of a coaxial cable, and a connector to distribute energy to each aperture antenna in the array via microstrips. Both the number and type of antennae that can be selected for use can be varied from that shown in order to, for example, vary the depth of energy penetration into the skin or tissue and/or to vary the size or area of focused energy emitted to skin or tissue.

FIG. 4 shows an embodiment of the invention incorporated into a computer controlled system for emitting energy from various directions into tissue, such as muscle tissue. There are four energy or microwave emitting handheld units (15) connected to the unit through cable (16). One or more units can be placed in proximity to a desired tissue. The handheld units can monitor the temperature of the tissue or biological sample that is being treated by the system and relay the information to control the emission of microwaves. Control parameters can be displayed on a monitor (17).

FIG. 5 depicts several examples of the antenna designs possible for emitting energy into skin or tissue. The design of A represents a multi-patch antenna having four patches (20) embedded or associated with a substrate and connected via patch distribution (19). The designs of B, C and D depict exemplary annular slot configurations. In each, the substrate (18) can be selected from a variety of appropriate materials and can be firm or solid, or flexible. In B, C and D, the link to the power source (28), ground connection (29), and one or more annular rings are shown. Antenna feed is shown at (21). The design selected is largely dependent upon the type of energy being emitting, the area into which energy is emitted, the desired focal point size or area, and/or the frequency selected. Various combinations and sizes of annular slots or rings, (22) (23), (24), (25), (26) and (27), can be selected as known in the art.

FIG. 6 depicts a directional applicator or antenna for emitting energy in only one direction or from only one plane. In A, (31) represents the surface where the emitted energy is directed from and (30) depicts a blocking or shielding surface. A cable (32) connects to a probe within the interior of the cylindrical apparatus, where the probe is positioned at a desired position or distance (33) away from the blocking or shielding surface (30). The view in B is a cross-section of the same apparatus. The cable (32) connects to probe (35). A cylindrical gap region is formed around an inner area, where gaps (37) are shown formed from walls (38). The inner area is filled with appropriate material or resin (36) to allow the energy to be transmitted. The size of the inner area (36) can be selected for a particular probe or frequency or power source. The distance (39) and (40) can be selected for the particular probe, geometry and size of the gaps and inner area, and the thickness and material in the inner area and other components of the apparatus. Surface (34) prevents the propagation of waves, so that energy is emitting in only one direction, or at least substantially only one direction. This provides advantageous properties for a device that used by a person desiring energy to be emitted in a specific area or in only one direction. The distance (41) and (42) can be also varied to control the energy emitted and a focal point of emitted energy.

FIG. 7 shows an exploded view of a preferred configuration of an antenna apparatus or configuration of the invention. Surface (43) is opposite the side where the tissue or skin is contacted. Surfaces (45) and (46) are typically composed of the same material, however, in other embodiments different materials can be selected. Inner substrate (44) has components on one surface that form the annular aspects of the antenna, as shown in the FIG. 5, B, C and D. Guide wire (47) is on the opposite side of substrate (46). This configuration can be used in am antenna of FIG. 1, for example.

FIG. 8 depicts another view of a preferred embodiment or configuration of the antenna. The distance at (48) and (50) is calculated based upon the desired frequency of the antenna, the medium(s) through which the emitted energy will pass, such as the solid bolus surfaces, water, and skin or tissue, and the desired frequency at the point or area of energy action. For example, when the desired treatment is for skin, and a 5.8 GHz generator used and a focused energy area of approximately 4-5 mm in diameter is desired, an annular ring of diameter 9.2 cm can be selected and a frequency of approximately 860 MHz used in order to emit energy under the surface of the skin of approximately 433 MHz or 434 MHz, which can effectively denature or disrupt matrix proteins or connective tissue or fibers. The Rf supply or guide wire (49), typically copper, is connected to a 50 ohm transmission line or cable and can be positioned off-center by approximately 1.45 mm. Annular ring is also typically composed of copper and embedded or supported in a substrate having a defined dielectric constant compatible with the desired energy output and focal point desired. Typical substrate materials include plexiglass, a copolymer used as microwave laminates, or polyoxymethylene copolymer

FIG. 9 shows two views or diagrams of typical skin cross sections. Top drawing A shows the dermis-epidermis junction (DE junction), where in one embodiment the energy is focused in order to effect fibers or connective tissue or proteins. Bottom panel B shows a micrograph of the cross section of skin, where the external dermis is at the top layer, the external cells and cells of the epidermis are visible, and a marginal zone separates or identifies the junction between epidermis and dermis. Generally, in treating the skin for visible fine lines, wrinkles, and other age-related or sun-related defects, the area of focused energy is that region formed by the junction between the dermis and epidermis, or the region of concentrated connective tissue or collagen fibers. Effecting these areas may loosen or destroy the connective tissue or fibers and, therefore, prevent the surface from exhibiting lines or wrinkles.

FIG. 10 shows the predicted energy areas or focal points for two different antenna configurations, depicting the thermal effect of the microwave field on a tissue or material. The top (A) represents the energy predicted in one direction or from one plane with the antenna design as shown in FIG. 8. The bottom (B) depicts the energy predicted from the multi-patch design of FIG. 5 A. In both cases, the area of focused energy is entirely below the surface of the skin or surface of the apparatus when places on the external surface of skin and typically coupled via water to surface of skin. The area shown in (100) is energy of a certain intensity, the area of (101) is a more focused area of that energy, and the area of (102) is the approximate focal point of the energy. Similarly, the area shown in (53) is energy of a certain intensity, the area of (54) is more focused energy, and the area of (55) is the approximate focal point of the energy. Areas (51) and (52) show energy that would not be preferred for some of the embodiments of the invention. Thus, the treatment area can be directed below the surface of the skin or tissue to prevent surface heating or undesired surface effects.

FIG. 11 is an exemplary hand-held unit showing an internal device for moving the probe or antenna back and forth over the surface of a medium, such as skin or tissue. Rotating groove (56) forces the entire internal unit (57) to move back and forth. As stated, a preferred example moves 12.8 mm each pass, but other lengths can be selected based upon the medium or desired results. Surface (58) is where energy is emitted and is often in contact with the surface of the medium, preferably in contact with water or other solution to improve transmission.

FIG. 12 shows an underside view of the hand-held device of FIG. 11, where two separate elements are included. One of the elements can be a probe or antenna as described in FIG. 6 (60), and the other an ultrasound transducer (59) for imaging the skin or tissue. The microwave emission in combination with the ultrasound imaging can be used together to both visualize tissue and specific features of skin and tissue that are not easily detectable with ultrasound alone, and to pin point the placement of the emitted microwave energy under the surface of the skin or tissue.

FIG. 13 shows a similar view to that of FIG. 12, where the ring of the directional microwave antenna (60) can be seen, as exemplified in FIG. 6. Ultrasound transducer (59) and rod (61) for moving both transducer (59) and antenna (60) together over a surface are shown. Surface (62) can be covered or wetted to enhance transmission of ultrasound and microwave.

FIG. 14 depicts the energy output from an antenna similar to that shown in FIG. 8. The areas of different intensity, low (63), and high (64) and (65), can be seen. Images such as FIGS. 14 and 15 are used to optimize energy output and reduce the energy loss of the antenna design. They can also be used to calibrate energy output for an intended use.

FIG. 15 depicts the energy output for a multi-patch antenna similar to that shown in FIG. 3. Each patch (67) can be seen, and the area of highest intensity (66) can be seen.

FIG. 16 schematically represents the effect of modulated microwave pulses on ultrasound imaging. In top chart I, the peaks represent ultrasound pulses of, for example, 50 Mhz. Chart II represents the echoic results detected by the ultrasound detector. Chart III represents the amplitude modulation microwave energy. Chart IV represents the specific echo at the focal point of the microwave energy that is preferentially amplified and used to image tissue that would otherwise not be amplified.

FIG. 17 is a diagram of the microwave emitter device or antenna and its electronic and wave control components. The elements can be selected from those available in the art or known to one of skill in the art. The focused microwaves or energy or “Beam” is depicted below the waveguide antenna, which can be one as shown in FIG. 6.

FIG. 18 is a schematic diagram showing a fluid treatment device, such as a sterilizing device for blood plasma or other biological fluid. The tank (70) is generally made of a polymer material that can be heat sterilized and can be selected to be the same material as used in the substrate or surfaces of the antennae. The plasma or fluid can be seen at the top (74). Plasma or fluid in and out lines are labeled (72). Each side is comprised of one or more patch antennae, and one such antenna is shown circled by (73). The bolus water or fluid lines are shown as (71).

FIG. 19 is an expanded view of the patch antenna circled as (73) in FIG. 18. The antenna can be, for example, one of those shown in FIG. 5, 7, or 8. The bolus fluid inlet and output are shown (75) and coaxial connection (76). Each antenna, represented by the repeated rectangular areas, is similarly connected to coaxial cable and bolus fluid (not shown).

FIG. 20 is a graph of the fluid temperature inside the tank as shown in FIG. 18. The fluid is treated with microwaves for up to 30 minutes in this example and a series of temperature probes records the temperature throughout this time. The desired temperature here is approximately 60° C.

FIG. 21 depicts the arrangement of a multipatch antenna, where layer (80) is the surface to contact the skin or tissue to be treated, layer (81) is as shown in FIG. 22, layer (82) is as shown in FIG. 23, and layer (83) is as shown in FIG. 24. Combined with a flexible substrate or composition, the unit forms a flexible, multi-patch slot antenna for transmitting radiofrequency and more particularly microwave energy. This configuration displays advantageously consistent operation and efficiency of microwave generation into the tissue that is essentially independent of the curved configuration of the antenna (as shown by comparing FIGS. 25-27).

FIG. 22 depicts the micro-strip feed line oriented for a particular multi-patch antenna.

FIG. 23 depicts the upper printed double-slotted patches of an antenna.

FIG. 24 depicts the connection between the micro-strip feed line and coaxial cable, which is connected or soldered into a drilled rectangular metal box that is soldered between layers in a multipatch antenna.

FIGS. 25-27 show, on the left side axis, the return loss in dB, and at the bottom axis shown in 50 MHz increments the frequency from 184 MHz (left end) to 684 MHz (right end). The frequency at the center of the graph (dark arrow at bottom coinciding with center point) is 434 MHz, which is the desired operating frequency in this example. A return loss of −36 dB is equivalent to approximately 99.97% of the energy being radiated in the desired direction, and a return loss of −20 dB is equivalent to about 1% reflected and about 99% energy being directed to the desired direction. FIG. 25 shows a graph of the return power when a patch antenna, as shown in FIG. 21, is in a planar configuration (curving radius equal to infinity).

FIG. 26 shows a graph of the return power when the same patch antenna is in a curved configuration (curving radius equal to 1 wavelength in free space).

FIG. 27 shows a graph of the return power when the same patch antenna is in a second curved configuration (curving radius equal to 3 wavelengths in free space).

FIG. 28 depicts the arrangement of an exemplary coplanar patch antenna. A single silicone layer (92) has on one side a thin printed film with thin slots at area (90) and a central metallic (copper) patch (91) antenna area both fed by a coaxial cable connection at point (93). The antenna is designed to be used with the silicone substrate side facing the treatment tissue, and the silicone layer can be approximately 1.5 mm thick, with the entire antenna approximately 10 cm×10 cm.

### DETAILED DESCRIPTION OF THE INVENTION

Throughout this disclosure, applicants refer to texts and other sources of information. One skilled in the art can use the entire contents of any of the cited sources of information to make and use aspects of this invention. Each and every cited source of information is specifically incorporated herein by reference in its entirety. Portions of these sources may be included in this document as allowed or required. However, the meaning of any term or phrase specifically defined or explained in this disclosure shall not be modified by the content of any of the sources. Specifically incorporated by reference in their entirety are the disclosures of U.S. provisional application 60/668,059 filed on Apr. 5, 2005, 60/668,059, filed Apr. 5, 2005, and 60/676,298 filed May 2, 2005. The description and examples that follow are merely exemplary of the scope of this invention and content of this disclosure and do not limit the scope of the invention. In fact, one skilled in the art can devise and construct numerous modifications to the examples listed below without departing from the scope of this invention.

In general embodiments of the invention, an Rf or microwave energy emitting device is used, wherein the device is similar to that described above. Preferred energy wavelengths are above about 300 MHz, and 915 MHz can be preferred for embodiments complying with FCC guidelines for medical devices. Other ranges can be used, as noted above. The energy emitted can be focused to a desired point under the surface of skin or tissue by selecting the frequency transmitted from the generator, the power selected from the generator, the dielectric of the substrate used in the antenna, the impedance of the transmission lines, the configuration of the antenna and the material used in its component parts, as well as the size and presence of a solution-filled bolus and the solution used and the temperature of the solution used. In addition, the bioelectrical impedance or differing dielectric of certain tissues and skin samples, for example, should be considered in the design to optimize energy treatment to a particular tissue of sample of skin. In this way, the invention encompasses methods of using an Rf or microwave emitter to treat a variety of conditions or disease states. For example, skin can be treated to improve the appearance, reduce fine lines or wrinkles, reduce or eliminate age lines, reduce or eliminate lines or wrinkles around the eyes, lips, forehead, neck, cheeks, ears, or chin. Tissue below the surface can be treated to destroy or ablate certain cells or tissue, such as tumors, diseased tissue, blemishes, or scars. Muscle tissue can be treated to reduce pain, reduce inflammation, or treat sore or painful muscles. Fat cells or fatty tissue can also be targeted to reduce bulging or cellulite areas. In separate embodiments, the Rf or microwave emitter can be linked to a bath or solution that can be treated to remove, reduce or eliminate microbiological growth or presence, and reduce or eliminate bacteria or viruses. For example, circulating water can be treated in heating, cooling, ventilation or humidity control systems. Also, plasma can be treated to reduce or eliminate the presence of contamination from bacteria or viruses.

In one version of the microwave or Rf emitting device, the power supplied from the generator or transmitter can applied in a range of 0.1-10,000 Watts per aperture and preferably, in a range of 2-50 Watts per aperture, or more preferably 2-10 Watts per aperture, and/or less than 40 Watts per aperture. The power is applied either in short, high power pulses or preferably, at a continuous wave frequency, and at a frequency in the range of 300-5000 MHz or more, or in a range of 430-5000 MHz, and most preferably, at a continuous wave frequency of about 433 MHz or about 434 MHz, or about 866 MHz or about 915 MHz. Treatment is continued for a desired amount of time in accordance with the desired results, preferably, less than 1 second to 5 minutes for high power pulses, and more preferably, for durations of 30 minutes to 4 hours for moderate power, or for extended periods (e.g. overnight) at even lower average power levels. The differing treatment times and energy ranges reflect the differing options in selecting an emitter or antenna configuration and the desired depth of treatment.

As one of skill in the art understands the existing devices, a simple electrode or energy emitting device for use in tissue treatment or ablation is a conductive probe or needle having an non-insulated tip, typically comprising an electrode or conducting material. The probe is energized by an oscillating electrical signal of approximately 460 kHz. Energy is emitted radially from the tip and produces a spherical or ellipsoidal zone of heating depending on the length and shape of the exposed tip. Thus, the area of treatment surrounds the tip and includes tissue in contact with or near the tip as well as tissue at some distance to the tip. In general, heat is generated in the tissue from the electromagnetic field surrounding the tip. The volume of tissue being treated can be controlled, in some sense, by moving a selected length of non-insulated tip. The amount and duration of the energy delivery can be varied to control the volume of tissue being treated.

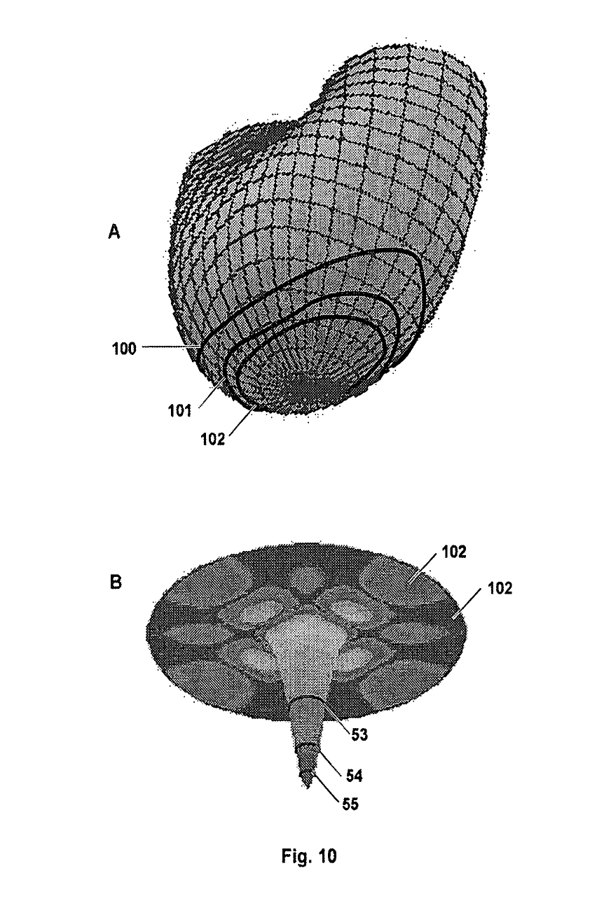

In certain embodiments of the invention, however, the tip is selected to be a radiofrequency (Rf) electrode or microwave antenna. As shown in FIGS. 10 A and B, the energy emitting from an appropriately designed antenna or device of the invention creates a focal point of treatment energy or heating effect, so that areas under the surface of skin or tissue can be treated non-invasively without damaging tissue at the surface of the skin. Advantageously, such a device and the use of it in the methods of the invention can reduce or eliminate undesired surface heating at the point of treatment or contact point with the skin or tissue. Surface heating and burns are severe problems in many devices currently available.

One particular embodiment of the device of this invention employs an ultrasound imaging technique or device to accurately position an RF electrode or microwave antenna directly near tissue or fibers to be destroyed. In a preferred embodiment combining the ultrasound device and a microwave emitter, the frequency of the microwave can be selected for a particular ultrasound pulse and tissue to create a contrast agent effect from the use of microwaves. In such embodiments, explained below, the ultrasound echo from tissue that will be selectively treated can be visualized through the ultrasound device when conventional ultrasound, without microwave treatment, will not allow the same visualization of the desired tissue. Thus, particular layers of skin at particular depths below the surface can be preferentially treated to improve treatment outcomes and/or reduce side-effects.

Other aspects of the invention will become apparent from the drawings and accompanying descriptions of the device and method of this invention. It will be readily apparent to a person skilled in the art that this procedure can be used in many areas of the body and many tissues of an animal.

The following Examples, and forgoing description, are intended to show merely optional configurations for the devices of the invention. Variations, modifications, and additional attachments can be made by one of skill in the art. Thus, the scope of the invention is not limited to any specific Example or any specific embodiment described herein. Furthermore, the claims are not limited to any particular embodiment shown or described here.

### EXAMPLE

### Cosmetic Dermatology and Muscle Treatments

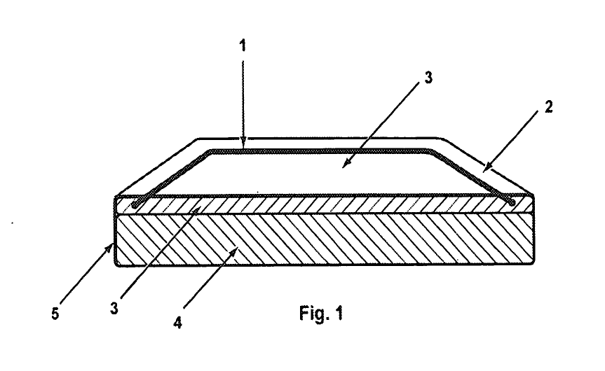

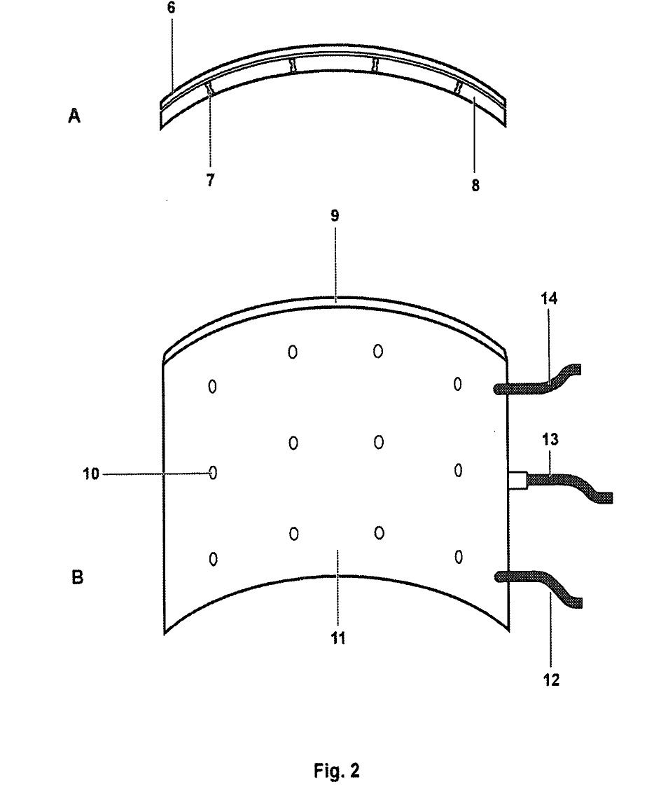

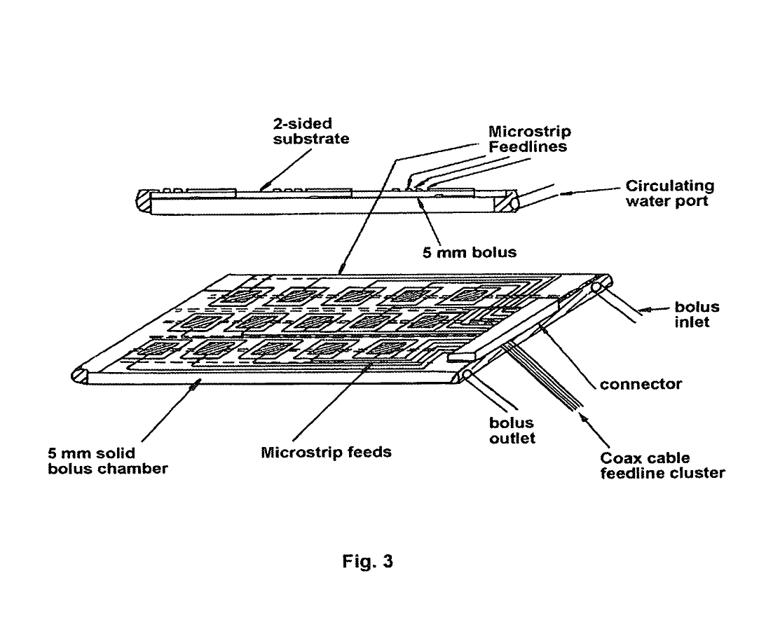

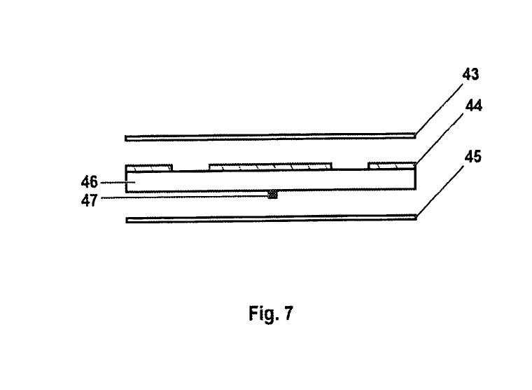

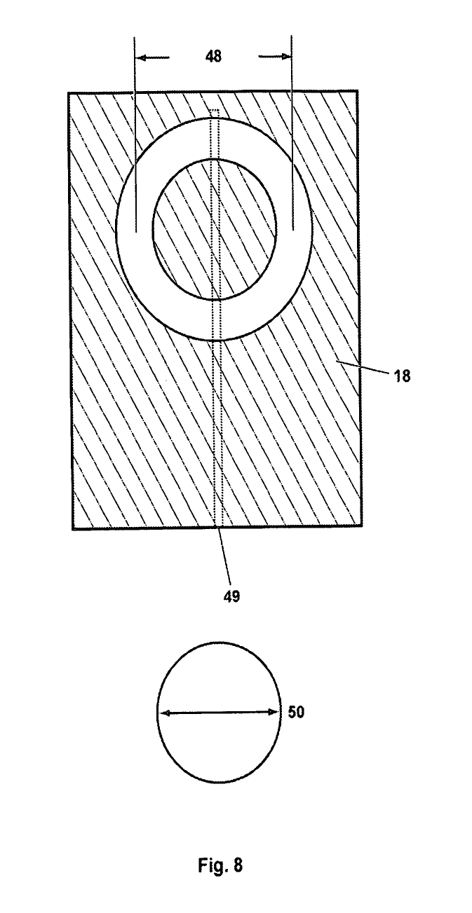

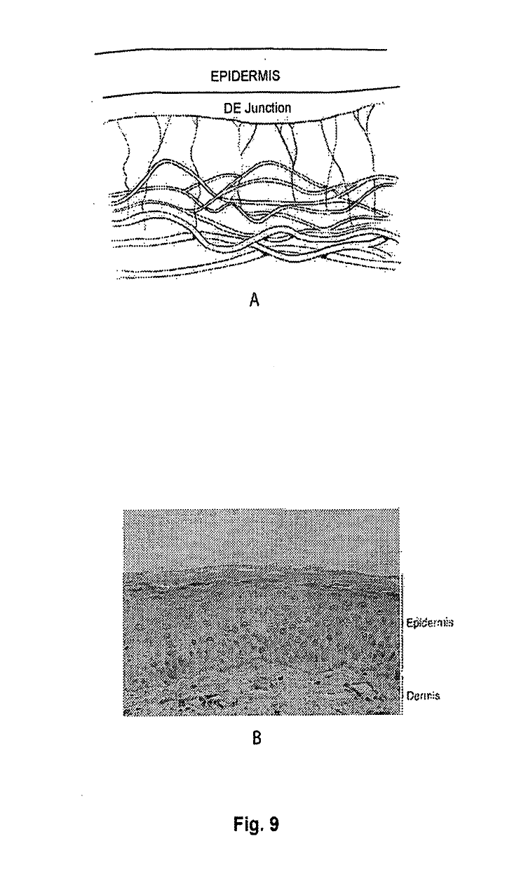

An example of the embodiment of FIG. 1, 2, 3, or 8 having a water-filled bolus and incorporating one or more elements of the invention is presented with the following characteristics. An antenna configuration is selected for the depth of treatment desired and energy or heating effect desired. For example, the annular ring configuration of FIG. 8 can be used, with the substrate formed of polyoxymethylene or plexiglass having a dielectric constant of approximately 3.5. By general convention, device is selected for 50 ohm transmitters, cables and guide wires, and microstrips, which are generally available. In a preferred embodiment, the generator and antenna are intentionally mismatched with respect to frequency to compensate for permittivity through bolus, bolus solution, and the particular skin or tissue. As shown in FIG. 8 for an annular slot antenna embodiment, the distance between the tops arrows (48) should be approximately one quarter the frequency (guided) used and in this example a 9.2 cm distance is used. The antenna has a copper surface with a ring inset allowing the substrate to form an annular ring, as shown in FIG. 7. The copper antenna feed is slightly off-center, by approximately 1.45 mm. Typically, a coaxial cable connection, ground point, and electrode are also present on the antenna (not shown). A solid water bolus chamber is placed on one surface of the antenna, the surface designed to be in contact with the skin or tissue, and preferentially fixed with a silicone adhesive. Usually water, distilled water, or saline is used in the bolus chamber. A fluid, preferably water, is generally used in between the bolus surface and the skin or tissue. The predicted shape of the energy emitted is shown in FIG. 10 A, which would focus heating energy to an area approximately 4-5 mm in diameter and at least 0.5 below the surface of the skin or tissue. In preferred embodiments, the water in the bolus is temperature-controlled to fine-tune the focusing of the energy area. A temperature of anywhere from about 17° C. to about 25° C. or about 37° C. can be selected, but other temperatures or ranges can be selected. Both the type of solution used in the bolus chamber and the temperature of the solution can be varied to affect the dielectric of this element of the device and therefore affect the shape and size of the focused energy and its depth or range of depths below the surface of the bolus.

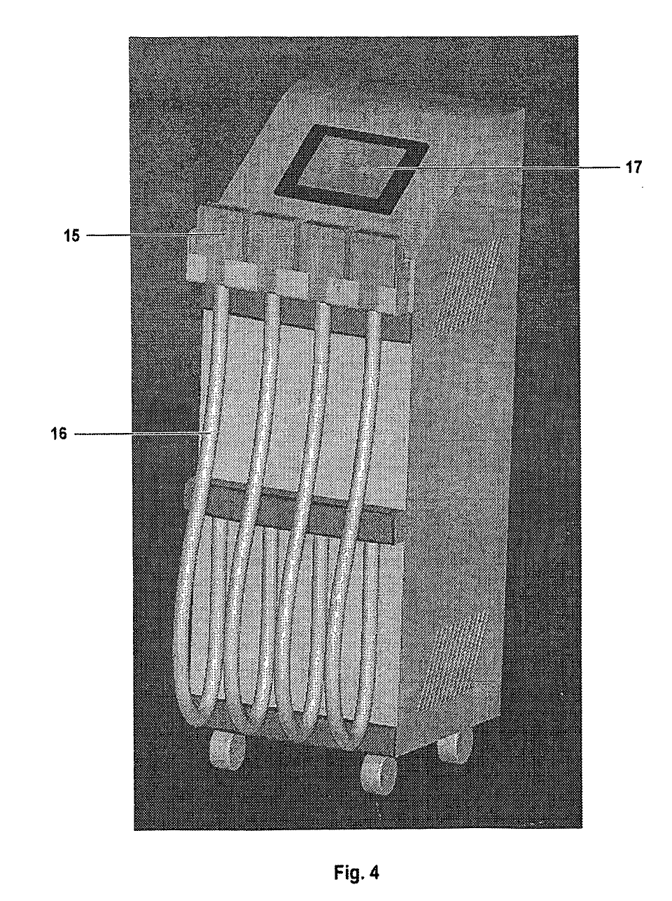

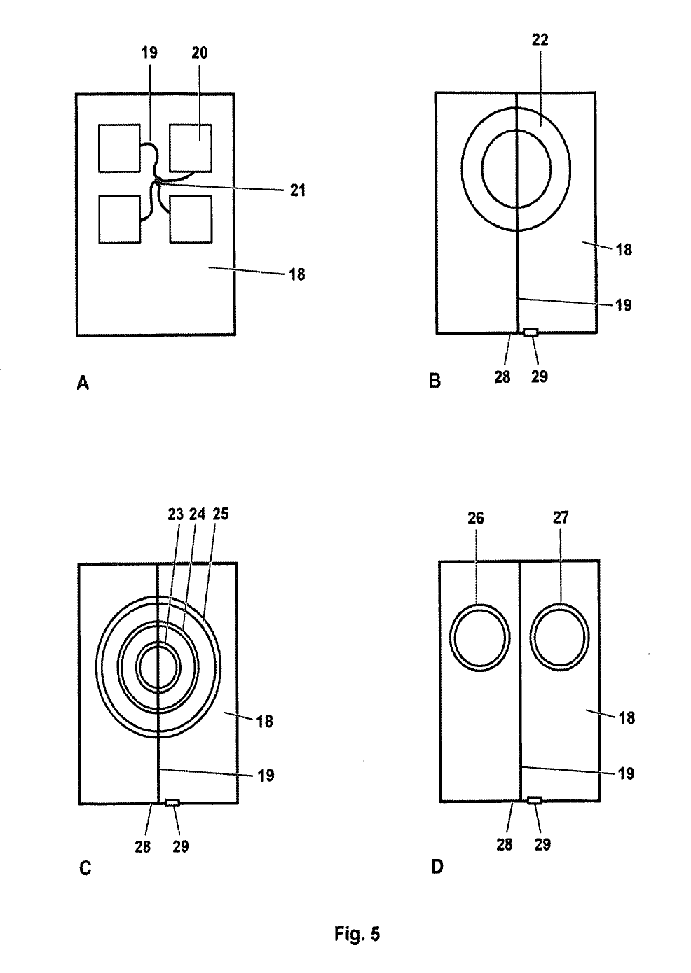

In one method of using the apparatus, the antenna or microwave emitter is connected to a 5.8 GHz generator via coaxial cables and the water bolus temperature set to a desired temperature between 17° C. and 25° C. The antenna is housed in a handheld applicator device similar to that depicted in FIG. 4. In FIG. 4, there are actually four hand-held antennas connected to a signal generator and controller. One or more of the hand-held antenna apparatus are placed in contact with skin around an area of muscle pain or tenderness or swelling. The power is turned on and the heating monitored. Placing more than one hand-held antenna device to surround the area can improve the treatment effects.

In another embodiment, a single antenna is used to treat the face or neck area. The hand-held antenna device is placed in contact with the skin to be treated and a solution is applied, generally water, to connect the surface of the bolus to the skin. This device is designed to be used for the treatment of skin and particularly the reduction or elimination of wrinkles or fine lines in the face of a patient. The treatment regimen consists of microsecond pulses at about 866 MHz designed to penetrate to a depth of about 1.5 to about 2.0 mm below the surface of the skin.

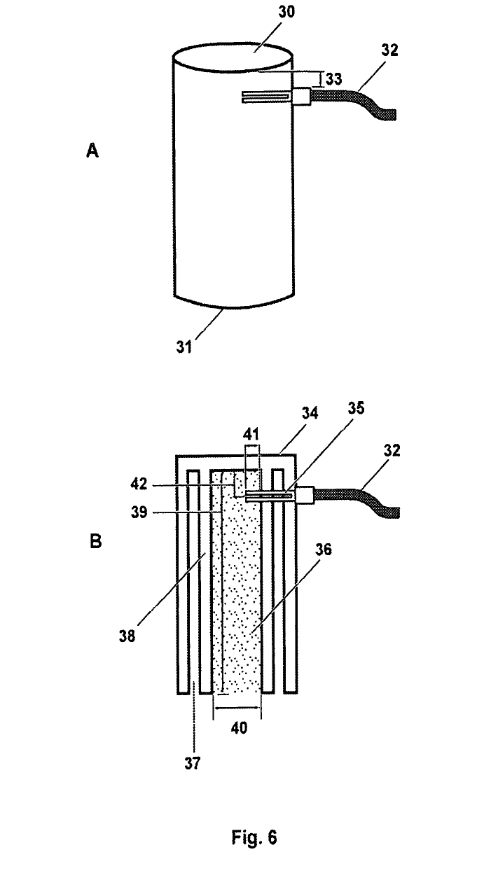

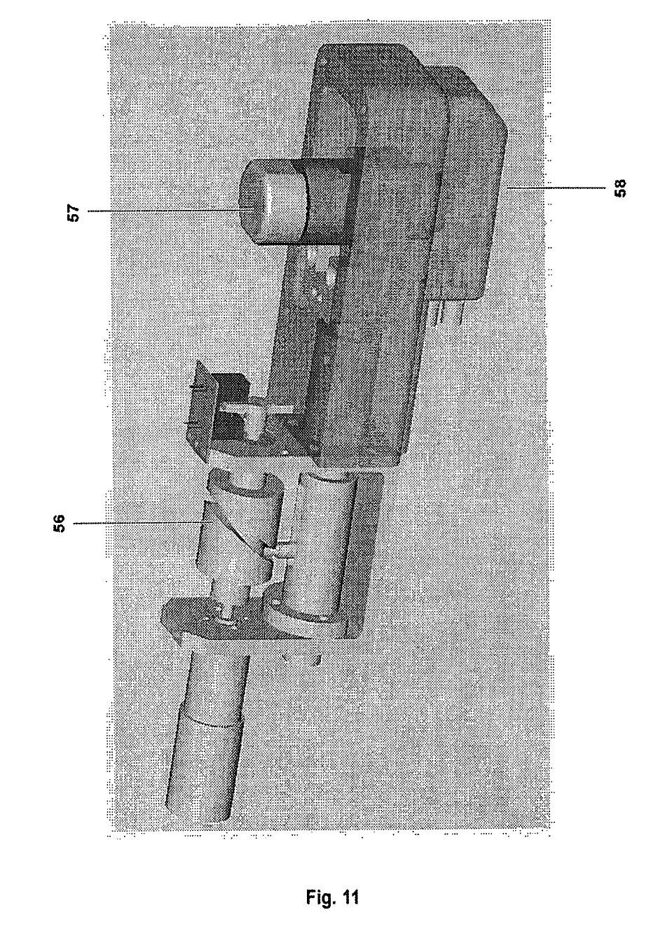

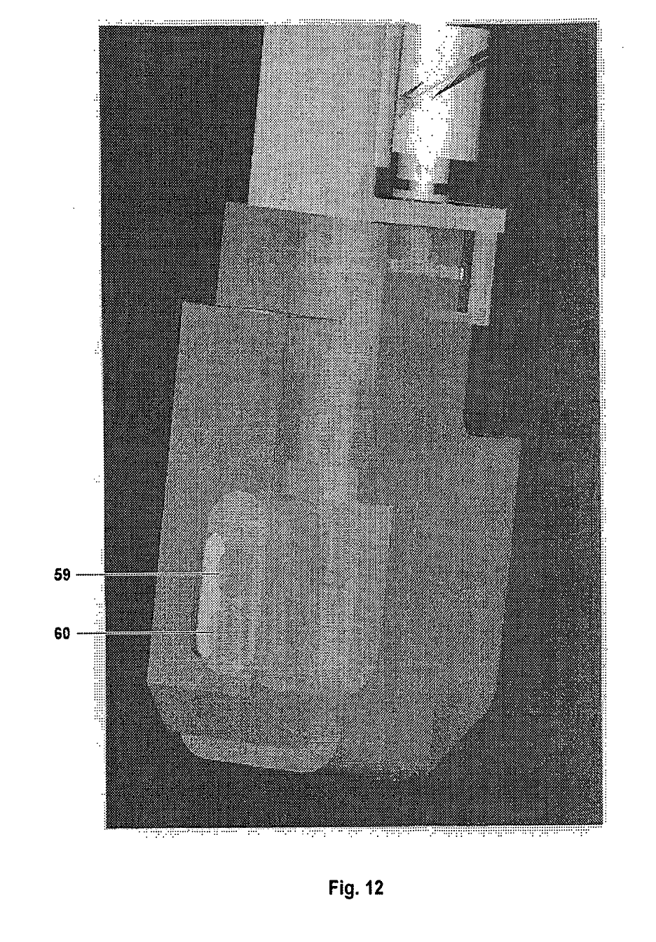

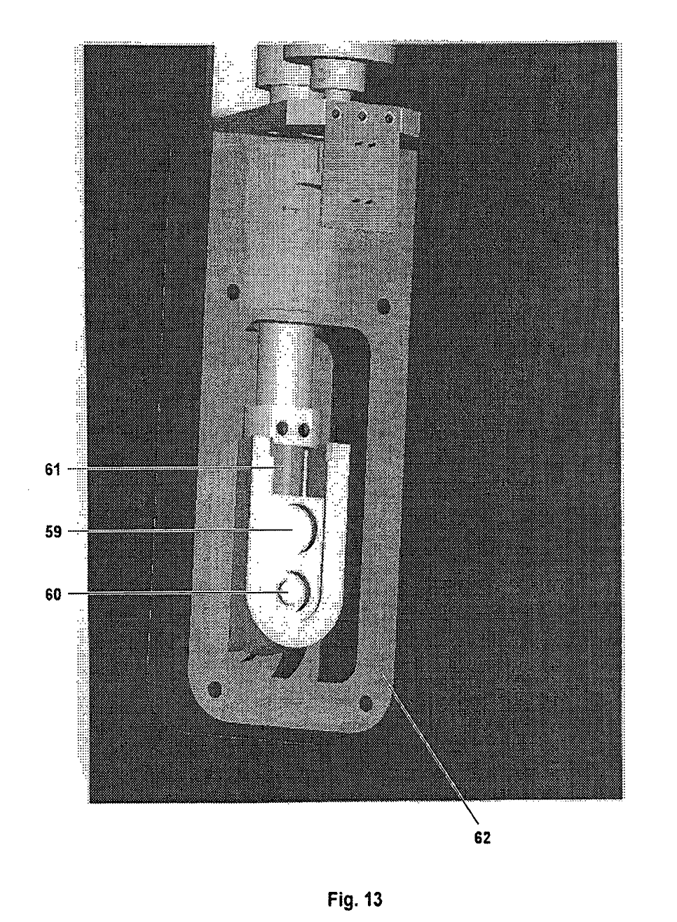

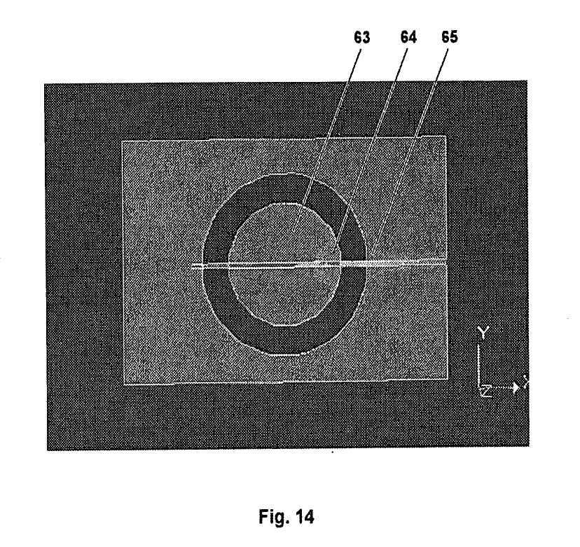

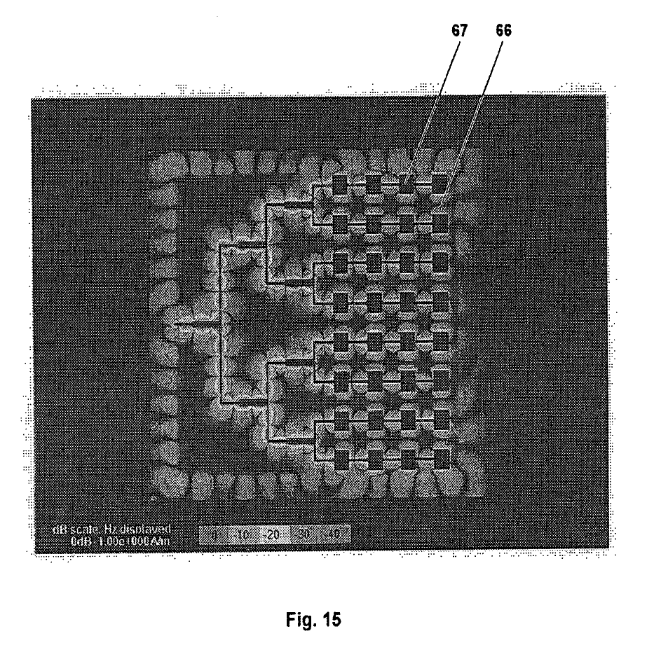

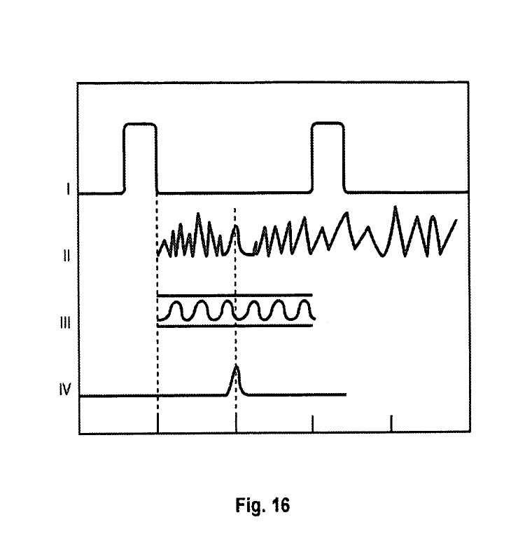

In another embodiment designed for cosmetic treatments of skin or other tissue treatments, a uni-directional antenna of FIG. 6 is used. This antenna employs a microwave short circuit surface or side to prevent the emission of energy on one side. Thus, the energy is solely emitted through the open, field-generating end through the dielectric resin of the interior chamber. Typically, the coaxial cable, again 50 ohm, is connected to a gold probe of a particular length and configuration and placed a particular distance from the end of the circular waveguide device, configured for 5.8 GHz in this example, although other frequencies can be selected. Printed gold or copper antenna designs and wires or connectors, as know in the art, can also be used. A field similar to that shown in FIG. 10 can be generated. This particular device of FIG. 6 is specifically useful when the field-generating end is either submerged in water or solution or sufficiently wetted to make a contact with the surface of the skin or tissue to be treated. The antenna device of FIG. 6 can be incorporated into a hand-held apparatus as shown in FIGS. 11, 12, and 13. In a preferred embodiment, the antenna device is coupled with an ultrasound probe to image the area being treated.

Fluid Treatment Systems

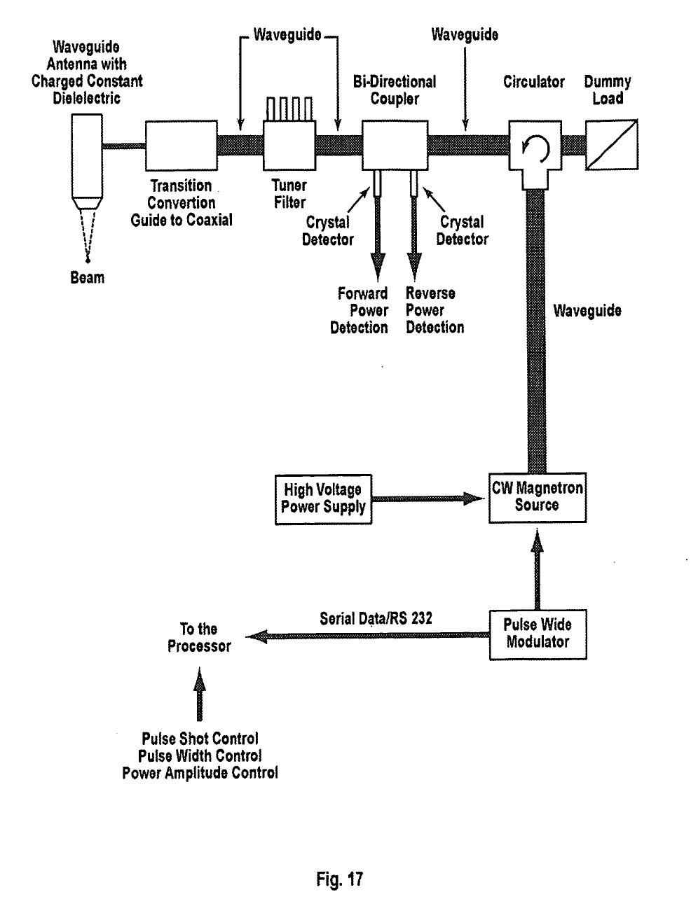

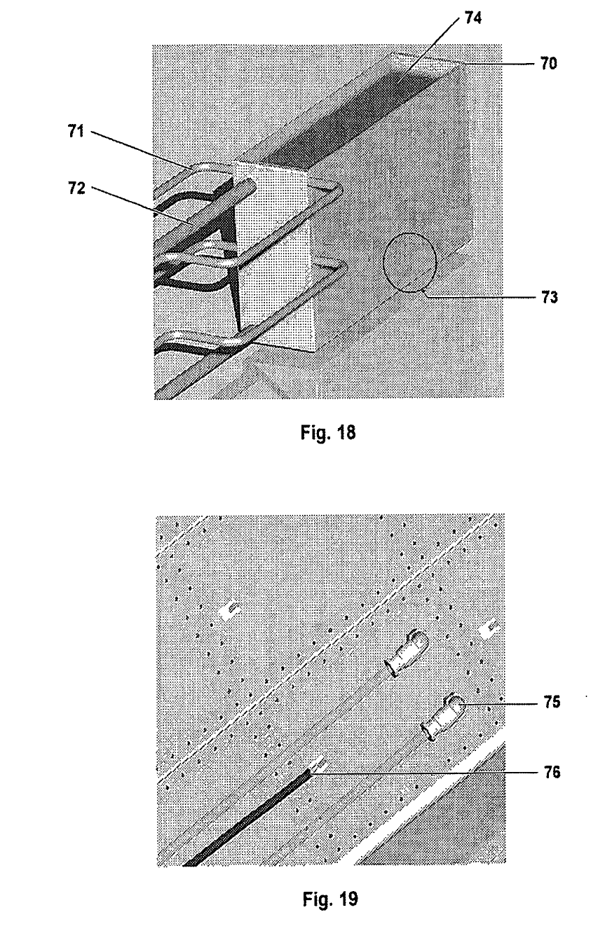

An embodiment designed for treating a fluid is shown in FIG. 17. A preferred embodiment is a device and method for sterilizing blood plasma or other biological fluids. The tank (70) is generally made of a polymer material that can be heat sterilized and can be selected to be the same material as used in the substrate or surfaces of the antennae. For a blood plasma device, a 50 liter tank can be used. The plasma or fluid can be seen at the top (74). Plasma or fluid in and out lines are labeled (72). Each side is comprised of one or more patch antennae, and one such antenna is shown circled by (73). The bolus water or fluid lines are shown as (71).

An example of the use of a 1 liter fluid volume shows that the temperature can be maintained at a specific sterilization temperature, or any desired temperature. The graph of FIG. 20 depicts the results for a 1 liter test using saline solution. The tank is composed of polyoxymethylene or plexiglass and 8 temperature probes are spaced within the tank to monitor temperature. For approximately 30 minutes, microwaves at approximately 434 MHz ere emitted into the tank with the bolus temperature of approximately room temperature. A single patch antenna can be used on each side of the tank, or multiple antennae on each side. A stirring mechanism or stir bar can be used inside the tank to circulate the fluid. As the graph of FIG. 20 shows, the temperature rises until the desired temperature is reached and then stabilized for the treatment period. For examples using blood plasma, a 60° C. temperature for about 10 or more hours can be selected and employed under sterile conditions. Other fluids and other temperatures and treatment periods can be used.

Tissue and Muscle Treatment Devices and Systems

In another embodiment especially suited for therapeutic treatment of living tissue, an antenna design incorporates the known or available information on the specific interaction possible between electromagnetic energy or microwave energy and living tissue, which depends on the nature of the tissue itself. For example, fat, muscle, skin, certain organs, each possess properties or characteristics that makes them slightly different from a treatment perspective. Measurements of actual or presumed permittivity data for various tissue is available and can be used.

In a particular embodiment of the invention, a microwave radiating patch antenna element can be developed with a double printed layer in order to irradiate different types of living tissue, such as muscle, bone, tendon, or fat. The radiating element used can be designed from flexible components, so that efficient contact with the tissue or the surface of the skin can be achieved. The actually permittivity of the tissues can vary, and preferred embodiments take into account the permittivity of about 9 for fat to about 30 for skin or to about 50 for bone at microwave frequencies. Because of the relatively high permittivity values of living tissues, the radiating element can be very small and it is possible to increase the slot width, in a slot aperture antenna, to a size capable of being printed on two metal patches. An example is shown in FIG. 21. A second printed support layer includes the micro-strip feed line as shown in FIG. 22. Since the feed line goes under the slot in the FIG. 22, a magnetic coupling leads to the transfer of energy from the feed line to the aperture. Then an electrical field in the aperture excites the patch to irradiate energy. Of course, it is possible to print metal patches on a third layer, and this can be desirable for cases when the permittivity of the medium to be irradiated becomes too low. The substrate selected for use between the two or more printed layers can be solid (a ceramic for example) or flexible (a silicone for example).

In the embodiment shown in FIG. 23, a small metallic component is connected to the small patch via holes at the bottom and left side of the micro-strip feed line layer, and to the bottom and left side of the slot layer. This assures a strong connection between the printed layer and a feed line, such as a coaxial cable, as depicted in FIG. 23.

The techniques to measure efficiency of the microwave energy transmission for a particular design and for a particular antenna design coupled with a biological tissue are known in the art. Furthermore, predicted fields for an antenna design can be generated by software models. By analyzing the efficiency, it becomes possible to design a radiating element to effectively radiate within a set of different materials with a permittivity from about 20 to about 50, for example, the permittivity of certain biological tissues. This can be optimized by adjusting the dimensions of the patch, the slot, and the thickness of the substrate used. When flexible components are exclusively used, the new design leads to a constant efficiency even when radiating element is placed into a curved or non-linear conformation. In addition, additional degrees of freedom are created by adding a new patch inside the slot. These new designs are particularly efficient for adjusting desired electromagnetic parameters.

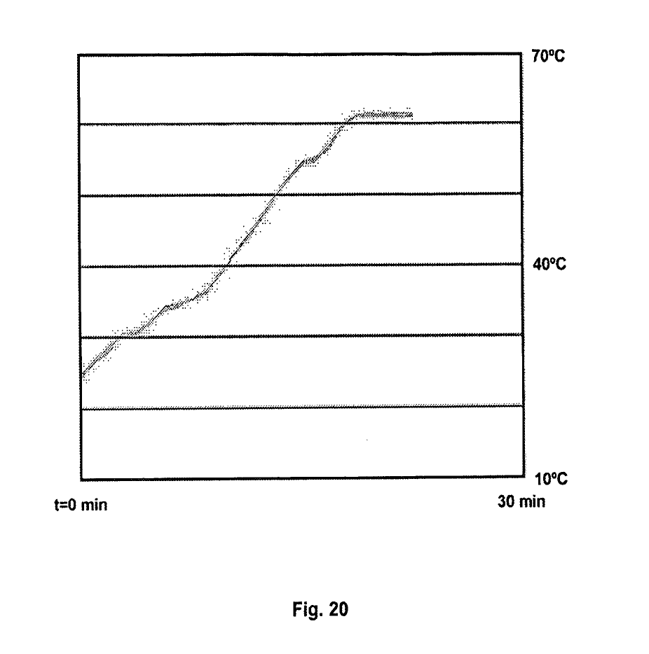

In a first set of examples, a flexible antenna designed as in FIGS. 21-23 are shown to have a radiating element capable of radiating energy in various materials of a permittivity range of at least 10 to 80 by adjusting the dimensions of the patch, slot and substrate thickness. This design leads a constant efficiency when the radiating element is curved to form over a body part or appendage. The data in FIGS. 25 to 27 show the consistent efficiency of the flexible antenna. Each figure represents the power that is not radiated. At the working frequency (central frequency shown in these graphs), reflected power remains below −20 dB, meaning more than 99% of the power is radiating to the desire surface or tissue.

A device and flexible antenna design for application to human muscle tissue, or the surface of skin over muscle, is prepared to deliver pulse or continuous wave energy in the range of about 434 MHz at about 100 mW/cm² maximum, using an amplifier of 35 joules. Four separate flexible antennae in planer silicone substrate (170 mm×130 mm) are formed into a sleeve or cuff-type device, for arm or leg, capable of inflating to enhance contact between antenna and skin. Each of the four antennae can be adjusted within the cuff to position them over desired areas. By applying to various body parts, tissue can be treated for a number of ailments and conditions, including but not limited to muscle contusions, muscle cramps, muscle pains, back pain, spinal or neck pain, muscle tears or strains, muscle fatigue, relaxing muscles, or for enhancing the recovery after strenuous work-outs. The treatments can be for less than 1 minute or several minutes, and can include multiple treatments over a period of time. For sports related treatments and training, the treatments can be combined with lactic acid monitoring or testing methods to determine optimum treatments times of regimens for particular muscle groups and workouts.

In a related device, a set of similar flexible antenna can be positioned in a horse or veterinary blanket to cover one or more muscle groups. The system can be designed to treat an animal prior to training and/or post-training to improve muscle performance and improve recovery from workouts. In a particular embodiment, 36 different antennae at 434 MHz are used to treat multiple major muscle groups of a horse. Various other monitoring devices, such as heart rate, oxygen and CO₂ sensors, and lactic acid monitoring, can also be used in conjunction with this an other embodiments. Effectively, the system can be used as a portable stress test and treatment device.

For these and other embodiments, various Rf frequencies within a broad range can be selected, and the 434 MHz is preferred, but others selected from the 915 MHz frequency band and microwave frequencies within the 2450 MHz frequency band approved for Industrial, Scientific, Medical (ISM) purposes can be selected. All of these Rf frequencies and microwave frequencies are suitable. If the generator selected operates at a certain fixed frequency X, the resonant frequency of the antenna may be tuned to this certain fixed frequency or tuned to a different frequency, such as anywhere between 0.5 X to 2 X, and about 2 X is preferred. Alternatively, the frequency of the generator used can be tunable and be adjusted to match the load impedance of the tissue being treated for particular antenna designs incorporating substrates of desired dielectric constants. For therapeutic purposes, the power selected for the energy pulse transmissions should be relatively high but not so high as to cause tissue damage. Generally, a safe temperature increase can be detected in the tissue being treated up to about 39° C. and below about 46° C. is often desirable. This temperature range can increase blood flow to the tissue. The use of a cuff or application device that conforms to the area desired to be treated minimizes any side effects of the irradiating treatments.

The substrate selected can be preferably a silicone, such as a KSIL from Silicone Engineering (Blackburn, Lancashire, UK). The dielectric constant of these silicones is about 2.9 and the thickness selected can vary, but a preferred thickness is 1.5 mm. As noted above, the selection of the substrate can incorporate the knowledge of the tissue designed to be treated and the treatment regimen, for example to account for impedance and permittivity at the contact point and changes in material, contact wetness, and/or whether or not water, aqueous solution or gel is used. Therefore, for contact against certain surfaces or body parts, one of skill in the art can select an appropriate combination of substrate, antenna, and generator operating conditions.

One skilled in the art can devise and create numerous other examples according to this invention. Examples may also incorporate additional imaging, thermometry, and other elements known in the art. One skilled in the art is familiar with techniques and devices for incorporating the invention into a variety of devices and of designing improved devices though the use of the concepts presented here.

What is claimed is:

1. A method for using an Rf or microwave emitting device, comprising
providing an emitting device comprising a multi-patch, annular antenna, slot antenna, or multiple-annular ring antenna formed within a flexible substrate and housed in an sleeve having an inflatable region, and further comprising a generator connected to a wave guide or wire linked to the antenna;
positioning the emitting device proximate to the sample or tissue under conditions in which the Rf or microwave energy can penetrate through the sleeve, and wherein the inflatable region can be inflated to apply pressure to one or more points of contact between an emitting surface of the antenna and the surface of the sample or tissue; and
creating one or more Rf or microwave pulses of about 1 msec in length and of a frequency and energy that penetrates to a desired depth of a sample or tissue,
wherein the energy penetrating the sample or tissue focuses on a predetermined region or area below a surface of the sample or tissue.

2. The method of claim 1, wherein the antenna is an annular slot antenna and the antenna and generator are mismatched relative to the frequency of operation.

3. The method of claim 2, wherein the antenna and generator are calibrated, tuned, or matched to frequency X and operated at a frequency between one half X and twice X, but not frequency X.

4. The method of claim 2, wherein the antenna and generator are matched to a frequency X and operated at a frequency of twice X.

5. The method of any one of claims 1-4, wherein the dielectric constant of the substrate is between approximately 2.5 and approximately 3.5.

6. The method of any one of claims 1-4, wherein the selection of the antenna, frequency, and substrate allow the Rf or microwave emitted to focus the energy of the pulses and heat an area of a sample or tissue at a depth of between 0.5 mm to about 16 mm below the surface of the sample or tissue.

7. The method of any of claims 1-4, wherein the tissue comprises muscle.

8. The method of any of claims 1-4, further comprising inflating the inflatable region, whereby the antenna or the substrate contacts skin proximate to muscle tissue to be treated.

9. The method of any of claims 1-4, wherein the antenna is formed into an array.

10. The method of any of claims 1-4, wherein the substrate selected is selected from a silicone polymer, a copolymer, and POM (polyoxymethylene) polymer.

11. The method of any of claims 1-4, wherein the tissue comprises muscle and further comprising monitoring one or more of lactic acid levels, oxygen levels, or CO₂ levels.

12. A microwave emitting device for treating skin comprising:
an antenna capable of emitting a directional, focused beam of energy, wherein the antenna is embedded in a flexible substrate having a selected dielectric constant, and wherein the substrate has a back surface and a front surface;
a solution-filled bolus attached to the substrate at the front surface of the substrate, wherein the solution is encased in a solid plastic having a desired dielectric constant and wherein the bolus is about 5 mm in thickness;
a power supply connected to the antenna and capable of sending appropriate power to the antenna to create a microwave pulse.

13. The device of claim 12, further comprising a thermometer capable of detecting the temperature of a sample or tissue in contact with the bolus.

14. The device of claim 12, further comprising a computer-controller for regulating the microwaves emitted in response to the temperature detected in the sample or tissue.

15. The device of claim 12, wherein the antenna is a λ/2 dipole antenna.

16. The device of claim 15, wherein the antenna is a dual concentric antenna.

17. The device of claim 15, further comprising multiple microwave antennae arranged in an array and connected in series.

18. The device of any one of claims 12-17, further comprising a temperature controller to regulate the temperature of the solution in the bolus.

19. The device of claim 12, wherein the frequency and energy of the microwaves being emitted from the antenna are selected based upon the permittivity of the selected substrate, the solution-filled bolus, the solution in the bolus, and the temperature of the bolus, whereby the emitted microwaves penetrate through the bolus and enter skin in contact with the bolus to a desired depth.

20. The device of claim 19, further comprising a skin temperature detector.

21. The device of claim 20, further comprising a computer-controller for adjusting the temperature of the bolus and the energy from the power supply in response to the skin temperature.

22. The device of claim 19, wherein the frequency and energy of the microwaves capable of being emitted from the antenna are selected based upon the permittivity of the selected substrate, the solution-filled bolus, the solution in the bolus, and the temperature of the bolus, whereby the emitted microwaves penetrate through the bolus and enter a fluid sample in contact with the bolus up to a desired depth.

23. The device of claim 22, further comprising a fluid temperature detecting device.

24. The device of claim 23, further comprising a computer-controlled device for adjusting the temperature of the bolus and the energy from the power supply in response to the fluid temperature.

25. An Rf or microwave emitting device comprising
a cylindrical waveguide having a closed end and a field-emitting end, a probe inserted into an internal area filled with a dielectric compound or resin, and the probe connected to a generator;
optionally a cylindrical trap surrounding the internal area and separated by a gap of a desired distance;
and an ultrasound transducer and receiver.

26. The device of claim 25, wherein the ultrasound transducer and receiver is positioned adjacent to the Rf or microwave emitting device.

27. A method of using a device of claim 26 or 27, comprising using the cylindrical waveguide to generate energy directed to the surface of skin or tissue to be treated, either under water or in the presence of sufficient water or solution to couple the surface of the skin to the waveguide.

28. The method of claim 27, wherein the energy is directed to skin to reduce wrinkles or fine lines.

29. The method of claim 28, wherein the skin is the face or neck and the device is used over areas having wrinkles, lines, fine lines, aged lines, or other dermatologic features.

30. The method of claim 27, wherein Rf or microwave energy is emitted to focus at a point at least about 0.5 mm below the surface of the skin.

31. A microwave emitting device for treating tissue or fluid, comprising:
an antenna capable of emitting a directional field causing a focused area of thermal effect in the tissue or fluid, wherein the antenna is formed on or embedded in a substrate having a selected dielectric constant, and wherein the substrate has a back surface and a front emitting surface, wherein the antenna design is selected from an annular slot, coplanar patch, or multipatch antenna;
a cover, sleeve, or holding device capable of temporarily fixing the front emitting surface of the antenna to a tissue or body part or a tank or tube filled with a fluid for treatment; and
a power supply connected to the antenna and capable of sending appropriate power to the antenna to create a microwave pulse that is directed into the tank.

32. A method of treating a muscle or skin tissue or a biological fluid comprising sending appropriate power to the antennae in the device of claim 31 for a pulse of a desired period of time.

33. The method of claim 32, wherein microwaves of approximately 434 MHz or 915 MHz are used.

34. The method of claim 32, wherein the fluid is blood or plasma.

35. A method of using an Rf or microwave emitting device, comprising
providing an emitting device comprising a cylindrical, multi-patch or multiple-annular ring antenna formed on or within a substrate, optionally having an aqueous solution-filled bolus on a surface of the antenna for contacting a sample or tissue, and a generator connected to a wave guide or wire linked to the antenna;
positioning the emitting device proximate to the sample or tissue under conditions in which the Rf or microwave energy can penetrate the tissue and optionally through the bolus; and
creating one or more Rf or microwave pulses of about 1 msec in length and of a frequency and energy that penetrates through the bolus and is capable of penetrating to a desired depth of a sample or tissue,
wherein the energy penetrating the sample or tissue focuses on a predetermined area below a surface of the sample or tissue and does not heat the surface of the sample or tissue in a substantially detectable manner.

36. The method of claim 35, wherein the temperature of the solution in the bolus allows the Rf or microwave emitted to focus the energy of the pulses and heat an area of a sample or tissue at a depth of between 0.5 mm to about 16 mm below the surface of the skin.

37. The method of any of claims 35-36, wherein the temperature of the solution in the bolus is between about 17° C. and about 28° C.

38. The method of claim 36, wherein the temperature selected causes the emitted Rf or microwave energy to penetrate to a depth of between about 1.5 mm to about 2 mm into the surface of skin.

39. The method of claim 35 or 36, further comprising circulating the solution in the bolus with a temperature controlled bath external to the bolus.

40. The method of any of claims 35 or 36, wherein the antenna consists of a configuration of approximately 8-10 um diameter concentric arrays.

41. The method of claim 35, wherein the tissue is skin and the emitted microwaves are directed to an area of skin to reduce the appearance of wrinkles or fine lines.

42. The method of claim 35, wherein the tissue is skin and further comprising measuring the temperature of the skin.

43. The method of claim 35, further comprising using an ultrasound image of the tissue to visualize the tissue being treated or desired to be treated.

44. A skin or muscle tissue treatment system comprising:
at least one flexible microwave antenna formed on or within a substrate having a skin contact side and an opposite side;
a computer-controlled generator for sending microwave pulses through a feed line to the antenna and monitoring the field produced as a result of the pulses;
wherein the frequency of the microwaves selected for the generator is mismatched relative to the frequency of the antenna, and the substrate is selected to allow microwaves to penetrate and treat skin or muscle tissue below the surface of the skin.

45. The system of claim 44, wherein the treatment frequency is about 434 MHz.

46. The system of claim 44, wherein the antenna fitted is into a sleeve, and wherein the sleeve further comprises an inflatable region on the opposite side of the substrate.

47. The system of claim 44, wherein the substrate is a silicone polymer.

48. The system of claim 44, wherein the antenna is capable of directing energy only to the skin contact side when the substrate and antenna are in a curved shape.

49. The system of claim 44, wherein the efficiency of the antenna is at least 90% at a selected frequency when comparing the planar conformation to a curved conformation.

50. The system of claim 44, wherein the treatment frequency is about 915 MHz.
